(19) United States
(12) Patent Application Publication
(10) Pub. No.: US 2024/0086596 A1
(43) Pub. Date: Mar. 14, 2024

(54) METHOD AND SYSTEM FOR PREDICTING THE PERFORMANCE OF BIOPHARMACEUTICAL MANUFACTURING PROCESSES

(71) Applicant: NATIONAL INSTITUTE FOR BIOPROCESSING RESEARCH AND TRAINING, Blackrock, Co. Dublin (IE)

(72) Inventors: Colin Clarke, Ashbourne, Co. Meath (IE); Jonathan Bones, Blackrock, Co. Dublin (IE)

(21) Appl. No.: 18/030,487

(22) PCT Filed: Oct. 7, 2021
(86) PCT No.: PCT/EP2021/077793

§ 371 Date: Apr. 5, 2023

(30) Foreign Application Priority Data
Oct. 7, 2020 (GB) 2015861.4

(51) Int. Cl. G06F 30/27

(52) U.S. Cl. CPC G06F 30/27

(57) ABSTRACT

The present invention relates to a method and system for predicting performance of biopharmaceutical manufacturing processes by measuring a parameter of the biopharmaceutical manufacturing process at a predetermined sampling frequency, wherein said parameter is measured for n number of runs of said process, sequentially joining the parameter data of the n number of runs in a single continuous time-series manner, to generate a transformed data, training a time series analysis model for forecasting performance of the biopharmaceutical manufacturing process, based on a plurality of transformed data generated corresponding to a plurality of parameters, measuring the plurality of parameters at a predetermined timepoint, and using said measurement to reinforce and improve the trained time series analysis model, and predicting the plurality of parameters for a future run of the biopharmaceutical manufacturing process, based on the trained time series analysis model.

### FIELD

The present disclosure relates to a method and system for predicting the performance of biopharmaceutical manufacturing and/or biomanufacturing processes.

### BACKGROUND OF INVENTION

In recent years, the biopharmaceutical industry has sought to apply a variety of multivariate statistical analysis methods and machine learning algorithms for deeper understanding of biopharmaceutical manufacturing processes, process development, as well as for predicting values of various quality parameters pertaining to the biopharmaceutical manufacturing process.

Major issues known in the art while employing such multivariate statistical methods and machine learning algorithms includes challenges involved in continuous acquisition of data from the plurality of unit operations constituting the biopharmaceutical manufacturing process, and the generation of a sufficiently high number of sample points to train machine learning algorithms for accurate prediction of future events. These issues arise in part due to the lack of availability of high-resolution analytical instrumentation systems for simultaneously measuring critical process parameters as well as obtaining critical quality attributes in real-time. While of late, instrumentation systems capable of making such measurements are being developed, harnessing their full potential requires suitable complex data analysis methods.

Methods known in the art for application of multivariate statistics and machine learning analysis in the biopharmaceutical industry, analyses data from a pre-defined number of distinct process runs and maintains the analyzed data in a two or three dimensional format. However, such known methods require generation of data from a large number of process runs and are often highly complex algorithms. Models developed some complex approaches are considered ‘Black box’. While “black box models” can be accurate their structure and operation is uninterpretable are consequently difficult to be detailed in simple language for regulatory approvals.

Machine learning algorithms known in the art for predicting performance of biopharmaceutical manufacturing processes often require a large, often impractical, training sample data set that is too expensive and/or takes too long to generate. Large training sample data sets are required by conventional machine learning algorithms to also decrease the risk of overfitting, that is, the risk of the model fitting the training data, but future unseen samples getting mis-classified.

A range of commercial software packages, such as ‘Unscramble’ and ‘JMP’, are also known in the art for analysis of biopharmaceutical manufacturing process data. However, said commercial software packages are not capable of maximizing the utility of high-resolution data captured continuously from a bioprocess and also do not utilize time-series analysis of Critical Process Parameters (CPP's) and Critical Quality Attributes (CQA's) to monitor and forecast performance. Further, time data is not directly utilized for data analysis, but is used to stratify available data, or is encoded as a categorical variable.

WO 2018/229802 relates to a method for predicting outcome of a process used for manufacturing a sample in a bioreactor. The model described in the description stack data from different batches vertically within a matrix. WO 2019100040 discloses a predictive model that can predict parameter concentrations in the future based on initial, measured concentrations and historical data. EP 3702439 discloses methods, a computer program and a process control device for determining a multivariate process chart. A document published in Journal of Biotechnology, vol 162, 2012, Huong Le et al., titled ‘Multivariate analysis of cell culture process bio-process data-lactate composition as process indicator’ support vector machine regression, in which partial least squares require data to be stacked horizontally. However, each of the above-mentioned documents utilize complex models and does not utilize time-series analysis to monitor and forecast performance.

There is therefore an unfulfilled and unresolved need in the art for an improved system and method for predicting performance of biopharmaceutical manufacturing processes, which is straightforward to interpret and does not require large amounts of data for operation and overcomes at least one of the above-mentioned problems.

### SUMMARY OF INVENTION

The present invention relates to a method and system for predicting performance of biopharmaceutical manufacturing and/or biomanufacturing processes, as set out in the appended claims, using a time series analysis model trained by sequentially merged values of a plurality of critical quality attributes and a plurality of critical process parameters measured at a predetermined sampling frequency.

In a preferred embodiment of the present invention, there is provided a method for predicting performance of a biopharmaceutical manufacturing process. The method includes measuring a parameter of the biopharmaceutical manufacturing process at a predetermined sampling frequency, wherein said parameter is measured for n number of runs of said process; sequentially joining the parameter data of the n number of runs in a single continuous time-series manner, to generate a transformed data that indicates parameter data over a time duration of the n number of runs; training a time series analysis model for forecasting performance of the biopharmaceutical manufacturing process, based on a plurality of transformed data generated corresponding to a plurality of parameters; measuring the plurality of parameters at a predetermined timepoint, and using said measurement to reinforce and improve the trained time series analysis model; and predicting the plurality of parameters for a future run of the biopharmaceutical manufacturing process, based on the trained time series analysis model.

In an embodiment of the present invention, the plurality of parameters includes a plurality of critical process parameters (CPPs) and a plurality of critical quality attributes (CQAs).

In an embodiment of the present invention, the transformed data enable capturing a seasonality aspect of the parameter data over the time duration of the n number of runs.

In a preferred embodiment of the present invention, a method for predicting performance of a biopharmaceutical manufacturing process is provided. The method comprises the first step of continuously measuring a plurality of critical quality attributes and a plurality of critical process parameters at a predetermined sampling frequency, for one or more unit operations of the biopharmaceutical manufacturing process. In an embodiment of the present invention, the measured critical process parameters include viable cell density, osmolality, qualitative analysis of at least one metabolite, and quantitative analysis of at least one metabolite, and the measured critical quality attributes includes titre, aggregation profile, charge variant analysis, hydrophobic interactions, hydrophilic interactions, and middle-up mass analysis. Middle up mass analysis also includes glycan analysis.

The measured values of each critical process parameter and each critical quality attribute, for each unit operation is sequentially merged to capture the trends associated with each unit operation. The values of each critical quality attribute and each critical process parameter at a predetermined timepoint is then measured. Further, a time series analysis model is continuously trained (or reinforced) using the sequentially merged values of each critical process parameter and each critical quality attribute, and the values of each critical process parameter and each critical quality attribute at a predetermined timepoint. The time series analysis model could be a multivariate time series analysis model or a univariate time series analysis model. In an embodiment of the present invention, the multivariate time series analysis model used is a vector error correction model. The trained time series analysis model is used to predict future values of each of the critical quality attributes and each of the critical process parameters, for each unit operation.

In a preferred embodiment of the present invention, a system for predicting performance of a biopharmaceutical manufacturing process is provided. The system comprises a memory means operatively coupled to a computing device. The memory means has a plurality of instructions stored thereon which configures the computing device to continuously measure a plurality of critical process parameters and a plurality of critical quality attributes at a predetermined sampling frequency for at least one unit operation of a biopharmaceutical manufacturing process. The computing device is further configured to sequentially merge values of each of the measured critical quality attributes and critical process parameters, for each unit operation, and to measure values of each of the critical process parameters and critical quality attributes at a predetermined timepoint. The sequentially merged values and the measured values of the critical process parameters and the critical quality attributes at the predetermined timepoint, are used as inputs to continuously train a time series analysis model. The trained time series analysis model is utilized to predict values of each of the critical quality attributes and each of the critical process parameters, for each unit operation of the biopharmaceutical manufacturing process.

In a preferred embodiment of the present invention, there is provided a system for predicting performance of a biopharmaceutical manufacturing process. The system includes a variable measurement module that is configured to measure a parameter of the biopharmaceutical manufacturing process at a predetermined sampling frequency, wherein said parameter is measured for n number of runs of said process; a data transformation module configured to sequentially join the parameter data of the n number of runs in a single continuous time-series manner, to generate a transformed data that indicates parameter data over a time duration of the n number of runs; a data training module configured to train a time series analysis model for forecasting performance of the biopharmaceutical manufacturing process, based on a plurality of transformed data generated corresponding to a plurality of parameters, wherein the variable measurement module is configured to measure the plurality of parameters at a predetermined timepoint, and using said measurement to reinforce and improve the trained time series analysis model; and a performance prediction module configured to predict the plurality of parameters for a future run of the biopharmaceutical manufacturing process, based on the trained time series analysis model.

The present invention has wide industrial application and can potentially be used by all major biopharmaceutical companies for process development activities, knowledge enhancement, for monitoring commercial manufacturing, and for reverse engineering an existing product to optimise a cell culture bioprocess. The present invention can also be used with any platform or measuring system that acquires data at a given frequency from any unit operation of a biopharmaceutical manufacturing process, such for example, a cell culture process, a recombinant protein production process, Adeno-Associated Virus Production, or CAR-T cells manufacturing process, processes for making vaccines, viral vaccines and the like.

The present invention also allows scope for progressive refinement, that is the time series analysis model as per the present invention can be continuously reinforced and made more accurate with continuously generated new training data. The acquisition of multiple datapoints for each critical quality attribute and each critical process parameter and the subsequent merging of values maximizes the sample data set available for prediction analysis, which enables the model to get progressively more accurate.

Further, the present invention is easy to implement and is far less complex than machine learning models known in the art. The present invention is hence less prone to overfitting and is easily interpretable which in turn enables biopharmaceutical companies to easily comply with applicable regulatory laws.

There is also provided a computer program comprising program instructions for causing a computer program to carry out the above method which may be embodied on a record medium, carrier signal or read-only memory.

The present invention overcomes the problems associated with previously used machine learning approaches by joining the data from end of one batch to the start of another to capture the trends associated with a unit operation and enabling the use of well-established univariate or multivariate timeseries analysis methods for knowledge generation, process monitoring and forecasting. Also, acquisition of multiple datapoints for each variable over the course of the process and the subsequent joining of the process data maximises the sample number available for analysis. Many time series analyses are far less complex than newer machine learning approaches, making them less prone to overfitting and crucially interpretable making the path to regulatory acceptance clearer for biopharmaceutical companies. The present invention hence provides a robust solution for problems identified in the art.

### BRIEF DESCRIPTION OF DRAWINGS

The present invention will be more clearly understood from the following description of embodiments thereof, given by way of example only, with reference to the accompanying drawings, in which:—

FIG. 1 illustrates a system for predicting performance of biopharmaceutical manufacturing processes, in accordance with an embodiment of the present invention;

FIG. 2A illustrate the data acquired by the variable measurement module of system of FIG. 1, in accordance with an embodiment of the present invention;

FIG. 2B illustrates the transformed data generated by the data transformation module of system of FIG. 1, in accordance with an embodiment of the present invention;

FIG. 2C illustrates a graphical representation of the transformed data, in accordance with an embodiment of the present invention;

FIG. 3 is a graphical representation illustrating prediction of a plurality of critical quality attributes using a preferred embodiment of the present invention; and

FIG. 4 is a flowchart illustrating a method for predicting performance of biopharmaceutical manufacturing processes, in accordance with an embodiment of the present invention.

### DETAILED DESCRIPTION OF DRAWINGS

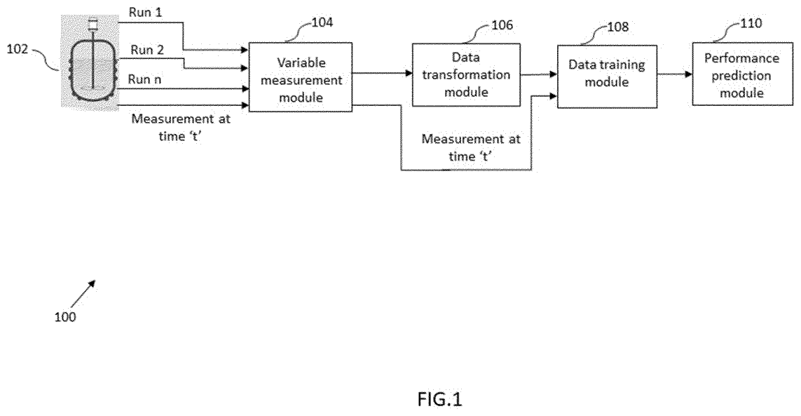

FIG. 1 illustrates a system 100 for monitoring and predicting performance of biopharmaceutical manufacturing processes in a bioreactor 102, in accordance with an embodiment of the present invention. Biopharmaceutical refers to a product produced by a cell in culture in the bioreactor 102. The bioreactor 102 comprises a cell culture medium and producer cells configured to product the biopharmaceutical product. Examples of such cells or producer cells include but is not limited to prokaryotic cells such as bacteria and eukaryotic cells such as Chinese Hamster Ovary (CHO) cells and Human Embryonic Kidney (HEK) cells. Examples of biopharmaceutical products includes monoclonal antibodies, bispecific antibodies, multispecific antibodies, antibody drug conjugates, proteins, blood factors, thrombolytic agents, hormones, interferons, haematopoietic growth factors, and nucleic acid-based biopharmaceutical products such as gene therapy drugs, treatments, products and the like.

The system 100 includes a variable measurement module 104 that is configured to measure variable parameters pertaining to manufacturing process, during each run of the bioreactor 102. In the context of the present invention, a run corresponds to an end to end biopharmaceutical manufacturing process. Throughout the invention, the run is interchangeably referred to as batch, and vice versa. Biopharmaceutical manufacturing is performed in batch or semi-continuous modes. All processes have a defined endpoint where the process is stopped. After a period of down-time, the process begins again from the start. Thus, the data produced via measuring can be considered to be a discontinuous, repeated time-series.

In an embodiment of the present invention, the biopharmaceutical manufacturing process is a cell culture process, and variable parameters may include characterisation of the bioreactor environment and aspects of the manufactured product such as yield and quality. Herein, the variable parameters may be of two types, Critical Process Parameters (CPPs) and Critical Quality Attributes (CQAs). Examples of a CPP include, but are not limited to, viable cell density, osmolality, qualitative analysis of at least one metabolite, and quantitative analysis of at least one metabolite. Examples of a CQA include, but are not limited to, titre, aggregation profile, charge variant analysis, hydrophobic interactions, hydrophilic interactions, and middle-up mass analysis.

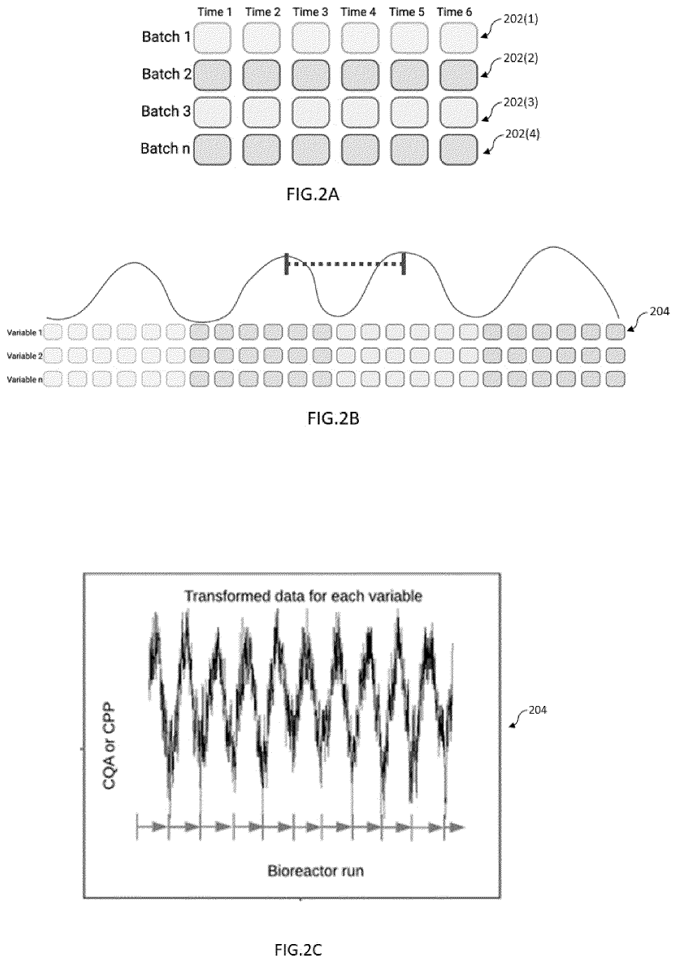

In an embodiment of the present invention, the variable measurement module 104 measures each parameter at a predetermined sampling frequency for a plurality of batches of the bioreactor. Thus, during each batch, the variable measurement module 104 acquires a plurality of samples for each CPP or CQA. For example, referring to FIG. 2A, for a given variable, during first batch/run, a first set of samples 202(1), during second batch/run, a second set of samples 202(2), during third batch/run, a third set of the samples 202(3), and during nth batch/run, an nth set of multiple samples 202(n) are acquired.

Referring back to FIG. 1, the system 100 further includes a data transformation module 106, that is configured to transform the parameter data in vertical form (as shown in FIG. 2A) into a horizontal form (as shown in FIG. 2B). For each variable, the data transformation module 106 sequentially joins parameter data of first to nth batches to generate transformed data. For example, referring to FIG. 2B, the data transformation module 106 sequentially joins the first, second, third, and nth set of samples 202(1), 202(2), 202(3) . . . 202(n) to generate transformed data 204 for a given parameter. The transformed data 204 indicates parameter data over a time duration of the n number of runs, and is a single time-series that maintains time-resolved patterns in data, and enable the application of time-series analysis to the biopharmaceutical manufacturing processes. Thus, the inventive step of the present application is the data transformation that unfolds the data into a single horizontal row of data such as the transformed data 204.

FIG. 2C illustrates the transformed data 204 for a given parameter across multiple bioreactor runs, in accordance with an embodiment of the present invention. The transformed data 204 forms a time-series that maintains time-resolved pattern in data and enable the application of time-series analysis to process and predict future values of one or more variable parameters. The transformed data 204 also enable to capture of aspects such as seasonality for a given variable, and also help in establishing a trend of a given variable over a time period.

Referring back to FIG. 1, the data training module 108 uses a plurality of transformed data for a plurality of parameters as training sample data to train a time series analysis model. The time series analysis model could be a multivariate time series analysis model or a univariate time series analysis model. Examples of univariate and multivariate time series analysis models that could be used for the purposes of the present invention include ‘Autoregression, autoregressive moving average’, ‘Autoregressive Integrated Moving Average (ARIMA)’, ‘Seasonal Autoregressive Integrated Moving-Average (SARIMA)’, ‘Seasonal Autoregressive Integrated Moving-Average with Exogenous Regressors (SARIMAX)’, ‘Vector Autoregression (VAR)’, ‘Vector Autoregression Moving-Average (VARMA)’, ‘Vector Error Correction Model (VECM)’, and ‘Vector Autoregression Moving-Average with Exogenous Regressors (VARMAX)’. Thus, established univariate and multivariate algorithms that have been traditionally used in areas such as weather-forecasting and finance may be utilised for performance prediction in biopharmaceutical manufacturing processes.

It may be noted that without the specific transformation described by the data transformation module 106, the bioprocess datasets 202(1), 202(2), 202(3) . . . 202(n) are incompatible with well-established time series methods. The transforming of bioprocess data from multiple batches by joining each batch together as a single continuous time-series, enables the use of traditional time series analysis methods for understanding the biopharmaceutical manufacturing process and forecasting future performance of such process.

The trained time series analysis model is used to predict future values of each of the CPP's and CQA's, for each of the unit operations of the biopharmaceutical manufacturing process. The variable measurement module 104 is further configured to measure one or more variables at time ‘t’ and provide the measured values to the data training module 108 to continuously reinforce and improve the time series analysis model.

The system 100 further includes a performance prediction module 110 configured to predict one or more variable parameters based on the trained time-series model. The performance prediction module 110 provides insights or prediction of future process performance of the bioreactor 102.

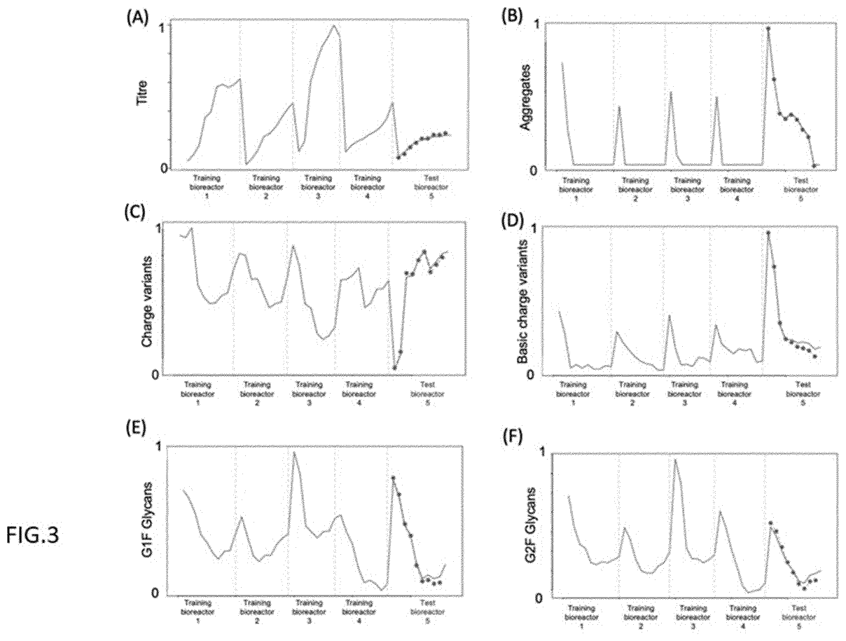

FIG. 3 is a graphical representation predicting cell culture performance in a recombinant therapeutic process in the bioreactor 102, using the system 100.

The variable measurement module 104 acquires data from the bioreactor for a CHO cell line producing a monoclonal antibody. The variable measurement module 104 employs an automated measurement system that characterises a range of measurements from the cell culture environment as well as critical measures of the recombinant protein. In total, 4 bioreactor runs are carried out and the data transformation module 106 joined the resulting data end to end in a manner as shown in FIG. 2B.

The data training module 108 employs a multivariate time-series analysis termed vector error correction model (VECM) to predict future performance to demonstrate the utilisation of the given approach with time-series analysis. An individual VECM time-series prediction model is trained using the CPP data (Lactate, Ammonia, Glutamine, Viable cell density, Viability and Osmolality) and a single CQA of interest. CPP data is used to predict the value of the CQA at each time point for the test bioreactor 24 hours in future. Upon reaching the 24-hour prediction, the measured data is used to reinforce the model and a new prediction is generated. In FIG. 3, in the test bioreactor event, the markers indicate each of the forecasted values and black line indicate the actual measurements of the CQA.

The forecasted values and the actual measurements demonstrate the accuracy of the novel data transformation and subsequent time-series analysis. The time-series model can be accurately be trained on as few as 4 batches reducing costs and development timelines. Crucially, the final models are interpretate and can be solved as an equation which can be presented to regulatory authorities during the approval process, as well as demonstrating the rationale regarding a process change during manufacturing.

In a preferred embodiment of the present invention, the system 100 may include a computing device and a tangible non transitory memory means operatively coupled to the computing device. The computing device may be a personal computer, a portable device such as a tablet computer, a laptop, a smart phone, connected household device or any operating system based portable device. The operating system deployed on the computing device may be Windows, OSX, Linux, iOS, Android, or the like. The memory means may be any internal or external device or web-based data storage mechanism adapted to store data.

The memory means has stored on it a plurality of instructions to configure the computing device to measure and process a plurality of CPP's and CQA's from one or more unit operations of the biopharmaceutical manufacturing process.

The computing device is configured to continuously measure CPP's and CQA's at a predetermined sampling frequency for at least one unit operation of a biopharmaceutical manufacturing process. The computing device is further configured to sequentially merge values of each of the measured CPP's and CQA's, for each unit operation, and to measure values of each of the CPP's and CQA's at a predetermined timepoint. The sequentially merged values and the measured values of the CPP's and the CQA's at the predetermined timepoint, are used as inputs to continuously train a time series analysis model. The time series analysis model could be a multivariate time series analysis model or a univariate time series analysis model. The trained time series analysis model is utilized to predict future values of each of the CQA's and each of the CPP's, for each unit operation of the biopharmaceutical manufacturing process. The system as per said embodiment utilizes the predicted values of CPP's and CQA's as training data to continuously reinforce the trained time series analysis model. The system further comprises a graphical user interface operatively coupled to the computing device and configured to graphically display the predicted values of each of the critical quality attributes and each of the plurality of critical process parameters, for each unit operation.

It will be appreciated that in the context of the present invention the system and method herein described can work in reverse where the CPPs can be predicted from the CQAs. One or more unit operations can be linked in a process such that the upstream parameters can be used to predict downstream. An advantage of this approach is that continuous manufacturing is enabled.

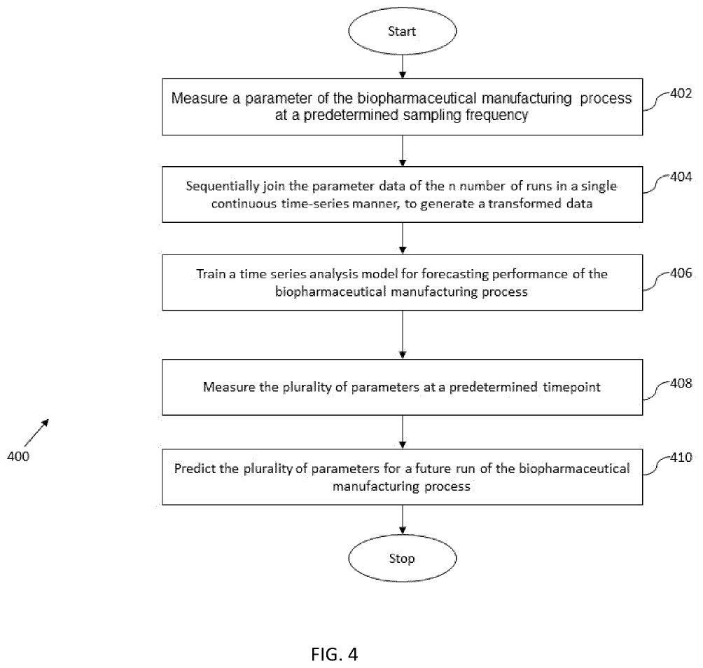

FIG. 4 is a flowchart illustrating a method 400 for predicting performance of a biopharmaceutical manufacturing process. At step 402, a parameter of the biopharmaceutical manufacturing process is measured at a predetermined sampling frequency, wherein said parameter is measured for n number of runs of said process.

At step 404, the parameter data of the n number of runs is sequentially joined in a single continuous time-series manner, to generate a transformed data that indicates parameter data over a time duration of the n number of runs.

At step 406, a time series analysis model is trained for forecasting performance of the biopharmaceutical manufacturing process, based on a plurality of transformed data generated corresponding to a plurality of parameters.

At step 408, the plurality of parameters is measured at a predetermined timepoint, and said measurement is used to reinforce and improve the trained time series analysis model.

At step 410, the plurality of parameters is predicted for a future run of the biopharmaceutical manufacturing process, based on the trained time series analysis model.

Although the present invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present invention as defined.

Further, a person ordinarily skilled in the art will appreciate that the various illustrative method steps described in connection with the embodiments disclosed herein may be implemented using electronic hardware, or a combination of hardware and software. To clearly illustrate this interchangeability of hardware and a combination of hardware and software, various illustrations and steps have been described above, generally in terms of their functionality. Whether such functionality is implemented as hardware or a combination of hardware and software depends upon the design choice of a person ordinarily skilled in the art. Such skilled artisans may implement the described functionality in varying ways for each particular application, but such obvious design choices should not be interpreted as causing a departure from the scope of the present invention. The method described in the present disclosure may be implemented using various means. For example, the system described in the present disclosure may be implemented in hardware, firmware, software, or any combination thereof. For a hardware implementation, the processing units, or processors(s) or controller(s) may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

For a firmware and/or software implementation, software code may be stored in the memory means and executed by a processor. The memory means may be implemented within the processor unit or external to the processor unit. As used herein the term “memory” refers to any type of volatile memory or non-volatile memory.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. a memory stick or hard disk. The carrier may be an electrical or optical signal which may be transmitted via an electrical or an optical cable or by radio or other means.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. A method for predicting performance of a biopharmaceutical manufacturing process, the method comprising the steps of:
measuring a parameter of the biopharmaceutical manufacturing process at a predetermined sampling frequency, wherein said parameter is measured for n number of runs of said process;
sequentially joining parameter data of the n number of runs in a single continuous time-series manner, to generate a transformed data that indicates parameter data over a time duration of the n number of runs;
training a time series analysis model for forecasting performance of the biopharmaceutical manufacturing process, based on a plurality of transformed data generated corresponding to a plurality of parameters;
measuring the plurality of parameters at a predetermined timepoint, and using said measurement to reinforce and improve the trained time series analysis model; and
predicting the plurality of parameters for a future run of the biopharmaceutical manufacturing process, based on the trained time series analysis model.

2. The method as claimed in claim 1, wherein the parameter data from an end of a run is joined to parameter data at start of next run.

3. The method as claimed in claim 1, wherein the plurality of parameters includes a plurality of critical process parameters (CPPs) and a plurality of critical quality attributes (CQAs).

4. The method as claimed in claim 1, wherein the time series analysis model is a multivariate time series analysis model.

5. The method as claimed in claim 4, wherein the multivariate time series analysis model is a vector error correction model.

6. The method as claimed in claim 1, wherein the time series analysis model is a univariate time series analysis model.

7. The method as claimed in claim 1, wherein the transformed data enable capturing a seasonality aspect of the parameter data over the time duration of the n number of runs.

8. The method as claimed in claim 1, wherein the plurality of parameters includes a plurality of critical process parameters that comprises viable cell density, osmolality, qualitative analysis of at least one metabolite, and quantitative analysis of at least one metabolite.

9. The method as claimed in claim 1, wherein the plurality of parameters includes a plurality of critical quality attributes that comprises titre, aggregation profile, charge variant analysis, hydrophobic interactions, hydrophilic interactions, and middle-up mass analysis.

10. The method as claimed in claim 9, wherein middle up mass analysis comprises glycan analysis.

11. The method as claimed in claim 1, wherein the biopharmaceutical is a monoclonal antibody.

12. The method as claimed in claim 1, wherein the biopharmaceutical manufacturing process is a cell culture process.

13. A system for predicting performance of a biopharmaceutical manufacturing process, the system comprising:
a variable measurement module configured to
measure a parameter of the biopharmaceutical manufacturing process at a predetermined sampling frequency, wherein said parameter is measured for n number of runs of said process;
a data transformation module configured to sequentially join parameter data of the n number of runs in a single continuous time-series manner, to generate a transformed data that indicates parameter data over a time duration of the n number of runs;
a data training module configured to train a time series analysis model for forecasting performance of the biopharmaceutical manufacturing process, based on a plurality of transformed data generated corresponding to a plurality of parameters, wherein the variable measurement module is configured to measure the plurality of parameters at a predetermined timepoint, and using said measurement to reinforce and improve the trained time series analysis model; and
a performance prediction module configured to predict the plurality of parameters for a future run of the biopharmaceutical manufacturing process, based on the trained time series analysis model.

14. The system as claimed in claim 13, wherein the parameter data from an end of a run is joined to parameter data at start of next run.

15. The system as claimed in claim 13, wherein the plurality of parameters includes a plurality of critical process parameters (CPPs) and a plurality of critical quality attributes (CQAs) .

16. The system as claimed in claim 13, wherein the time series analysis model is a multivariate time series analysis model.

17. The system as claimed in claim 16, wherein the multivariate time series analysis model is a vector error correction model.

18. The system as claimed in claim 13, wherein the time series analysis model is a univariate time series analysis model.

19. The system as claimed in claim 13, wherein the plurality of parameters includes a plurality of critical process parameters that comprises viable cell density, osmolality, qualitative analysis of at least one metabolite, and quantitative analysis of at least one metabolite.

20. The system as claimed in claim 13, wherein the plurality of parameters includes a plurality of critical quality attributes that comprises titre, aggregation profile, charge variant analysis, hydrophobic interactions, hydrophilic interactions, and middle-up mass analysis.

21. The system as claimed in claim 20, wherein top, middle or bottom up mass analysis also comprises glycan analysis.

22. The system as claimed in claim 13, wherein the biopharmaceutical is a monoclonal antibody.

23. The system as claimed in claim 13, wherein the biopharmaceutical manufacturing process is a cell culture process.

24. A method for predicting performance of a biopharmaceutical manufacturing process, the method comprising the steps of:
a) measuring a plurality of critical quality attributes and a plurality of critical process parameters at a predetermined sampling frequency, for at least one unit operation;
b) sequentially merging values of each of the plurality of critical quality attributes for each unit operation;
c) sequentially merging values of each of the plurality of critical process parameters for each unit operation;
d) measuring the plurality of critical quality attributes and the plurality of critical process parameters at a predetermined timepoint;
e) training a time series analysis model by inputting the merged values in step (b) and step (c), and the measured values in step (d); and
f) predicting values of each of the plurality of critical quality attributes and each of the plurality of critical process parameters, for each unit operation, by applying the trained time series analysis model.
